(19) United States
(12) Patent Application Publication
(10) Pub. No.: US 2009/0222013 A1
(43) Pub. Date: Sep. 3, 2009

(54) Tap Guide

(75) Inventors: Ben Graf, Madison, WI (US); Scott Freedman, Boston, MA (US)

Correspondence Address: NORMAN F. HAINER, JR.;SMITH & NEPHEW, INC., 150 MINUTEMAN ROAD, ANDOVER, MA, 01801, US

(73) Assignee: Smith & Nephew, Inc., Memphis, TN (US)

(21) Appl. No.: 12/396,141

(22) Filed: Mar. 2, 2009

Related U.S. Application Data
(60) Provisional application No. 61/033,106, filed on Mar. 3, 2008.

(51) Int. Cl. A61F 5/00

(52) U.S. Cl. 606/87

(57) ABSTRACT

In one aspect, the present disclosure relates to a tap guide including a shaft having a proximal portion and a distal portion, a handle coupled to the proximal portion of the shaft, and a tip coupled to the distal portion of the shaft. A method and system for preparing a bone tunnel for receipt of a fixation device is also disclosed.

### CROSS-REFERENCE TO RELATED APPLICATIONS

This application claims priority to United States Patent Application No. 61/033,106 filed on Mar. 3, 2008, the disclosure of which is incorporated herein by reference in its entirety.

### BACKGROUND

1. Field of Technology

The present disclosure relates to the preparation of a bone tunnel for receipt of a fixation device during ligament reconstruction surgery, and more specifically, a tap guide for use in such preparation.

2. Related Art

A ligament, such as an anterior cruciate ligament (ACL), that has ruptured and is non-repairable, is generally replaced arthroscopically by a soft tissue graft. The soft tissue graft can be harvested from a portion of a patellar tendon having so called “bone blocks” at each end, and from the semitendonosis and gracilis. Alternatively, the soft tissue graft can be formed from synthetic materials or from a combination of synthetic and natural materials.

The replacement soft tissue graft is implanted by securing one end of the soft tissue graft in a bone tunnel within the femur, and passing the other end of the graft through a tunnel formed in the tibia. The graft may be secured in the tunnels via the use of a fixation member, such as a screw, that is positioned between the wall of the tunnel and the tissue graft. While the fixation screw is being advanced through the tunnel, it is important that there be enough of a pathway to substantially reduce the possibility of screw breakage and graft rotation, yet allow enough contact for adequate screw fixation. Therefore, adequate preparation of the bone tunnel is required to achieve this goal.

### SUMMARY

In one aspect, the present disclosure relates to a tap guide including a shaft having a proximal portion and a distal portion, a handle coupled to the proximal portion of the shaft, and a tip coupled to the distal portion of the shaft. In an embodiment, the tip includes a groove having a tapered depth along a length of the groove. The groove is between about 40% to about 65% deeper at a second end of the groove relative to a first end of the groove. In another embodiment, the tip is located at an angle α; between about 2° to about 4°, relative to a longitudinal axis of the shaft. In yet another embodiment, the tip includes a diameter of between about 4 mm and about 12 mm. In a further embodiment, the shaft includes a through hole and a groove extending a length of the shaft. In yet a further embodiment, the proximal portion of the shaft includes a circular shape. In an embodiment, the handle includes a proximal portion and a distal portion wherein the distal portion is located at an angle relative to the proximal portion and the proximal portion is positioned substantially perpendicular to the proximal portion of the shaft.

In another aspect, the present disclosure relates to a method of preparing a bone tunnel for receipt of an anchor. The method includes providing a tap guide including a shaft having a proximal portion and a distal portion, a handle coupled to the proximal portion of the shaft, and a tip coupled to the distal portion of the shaft; providing a tap; inserting the tip into the bone tunnel; inserting the tap into a through hole of the shaft and into the bone tunnel to create a notch in a wall of the bone tunnel; and removing the tip from the bone tunnel. In an embodiment, the method further includes inserting an end of a tissue graft into the bone tunnel and inserting a fixation device into the bone tunnel via the notch, wherein the fixation device is located between the tissue graft and a wall of the notch. In another embodiment, the tip includes a groove having a tapered depth along a length of the groove such that during creation of the notch the tap cuts deeper in a first area of the notch than in a second area of the notch. In yet another embodiment, the diameter of the tip is substantially equal to the diameter of the bone tunnel. In a further embodiment, the notch is configured such that rotation of the tissue graft does not occur during insertion of the anchor into the bone tunnel.

In yet another aspect, the present disclosure relates to a system for preparing a bone tunnel for receipt of a fixation device. The system includes a tap; and a tap guide including a shaft having a proximal portion and a distal portion, a handle coupled to the proximal portion of the shaft, and a tip coupled to the distal portion of the shaft. In an embodiment, the tip includes a groove having a tapered depth along a length of the groove. In another embodiment, the groove is between about 40% to about 65% deeper at a second end of the groove relative to a first end of the groove. In yet another embodiment, the tip is located at an angle α, between about 2° to about 4°, relative to a longitudinal axis of the shaft. In a further embodiment, the tip includes a diameter of between about 4 mm and about 12 mm. In yet a farther embodiment, the shaft includes a through hole and a groove extending a length of the shaft. In an embodiment, the proximal portion of the shaft includes a circular shape.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the disclosure. In the drawings:

FIG. 1 shows a perspective view the tap guide of the present disclosure.

FIG. 2A shows a perspective view of the shaft of the tap guide of the present disclosure.

FIG. 2B shows a perspective view of the shaft and tip of the tap guide of the present disclosure.

FIG. 3A shows a perspective view of the tip of the present disclosure.

FIG. 3B shows a front view of the tip of the present disclosure.

FIG. 4 shows a perspective view of a tap of the present disclosure.

FIG. 5 shows a perspective view of the tap guide and tap during creation of a notch in a bone tunnel.

FIG. 6A shows a cross-sectional view of a bone tunnel and notch of the present disclosure.

FIG. 6B shows a perspective view of the bone tunnel and notch of FIG. 6A.

FIG. 7A shows a front view of a second area of a notch of a first embodiment of the present disclosure.

FIG. 7B shows a front view of a first area of the notch of FIG. 7A.

FIG. 8A shows a front view of a second area of a notch of a second embodiment of the present disclosure.

FIG. 8B shows a front view of a first area of a notch of a second embodiment of the present disclosure.

FIG. 9 shows a soft tissue graft and fixation device within the bone tunnel and notch of the present disclosure.

### DETAILED DESCRIPTION OF THE EMBODIMENTS

The following description of the preferred embodiment(s) is merely exemplary in nature and is in no way intended to limit the disclosure, its application, or uses.

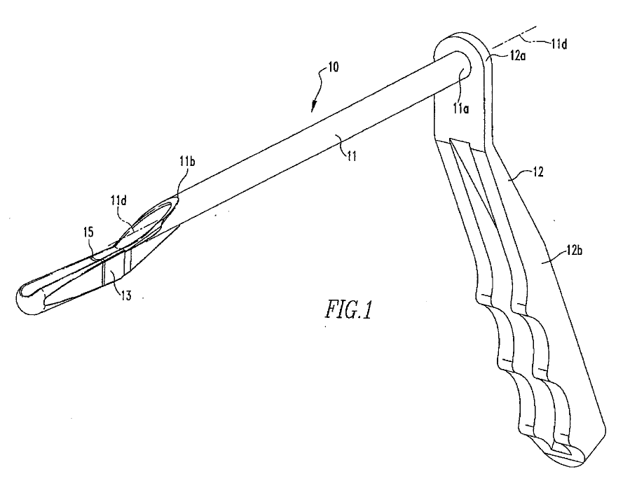

FIG. 1 shows the tap guide 10 of the present disclosure. The tap guide 10 includes a shaft 11 having a proximal portion 11a and a distal portion 11b, a handle 12 coupled to the proximal portion 11a of the shaft 11, and a tip 13 coupled to the distal portion 11b of the shaft 11. The handle 12 includes a proximal portion 12a positioned substantially perpendicular to a longitudinal axis 11d of the shaft 11 and a distal portion 12b located at an angle to the proximal portion 12a. The distal portion 12b is configured for providing a user with a proper grip during surgery, as will be further described below. It is within the scope of this disclosure that the proximal portion 12a may be positioned parallel, or at an angle to, the longitudinal axis 11d of the shaft 11. It is also within the scope of this disclosure that the distal portion 12b may be in-line with or not at an angle to the proximal portion 12a.

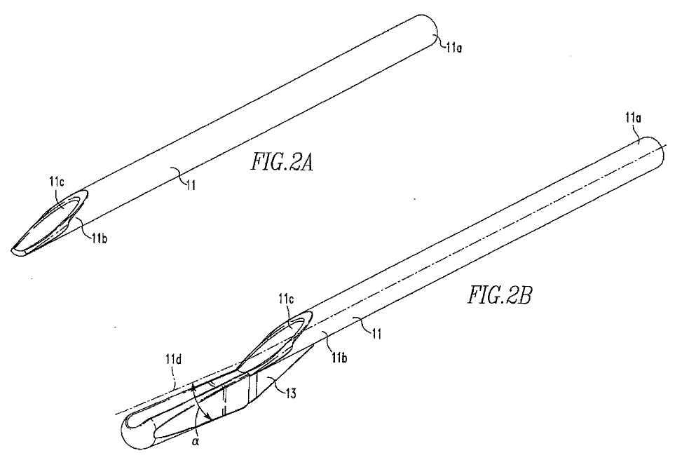

As shown in FIGS. 2A and 2B, the shaft 11 includes a through hole 11c extending a length of the shaft 11. The through hole 11c is configured for insertion of a tap, as will be more fully described below. Additionally, the shaft 11, including the proximal portion 11a, is substantially circular in shape. However, the distal portion 11b of the shaft 11 is beveled to facilitate coupling of the tip 13 to the distal portion 11b . The distal portion 11b may be beveled via machining or any other type of process. The tip 13 is located at an angle α, between about 2° and about 4°, relative to a longitudinal axis 11d of the shaft 11. However, for the purposes of this disclosure, the angle α may range from between about 0° to about 45°. Also for the purposes of this disclosure, the shaft 11 may be a shape other than circular.

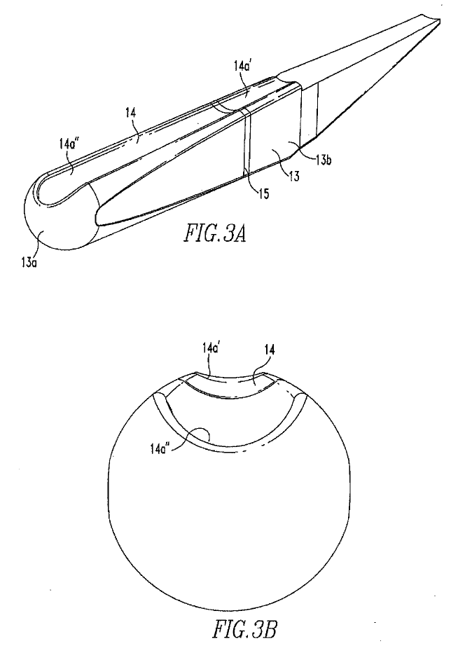

As shown in FIGS. 3A and 3B, the tip 13 includes a first end 13a, a second end 13b, and a groove 14 having a tapered depth along a length of the groove 14 such that the groove 14 is between about 40% to about 65% deeper at a second end 14a″ of the groove 14 relative to a first end 14a′ of the groove 14. The tip 13 also includes a laser mark 15 that will be used as a guide in controlling the insertion depth of the tip 13 and the tap into the bone tunnel, as will be further described below. The laser mark 15 is located between about 25 mm to about 35 mm from the first end 13a of the tip 13.

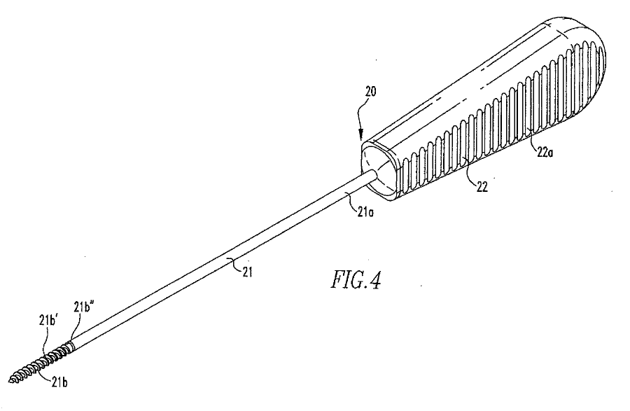

FIG. 4 shows a tap 20 that is used in cooperation with the tap guide 10 to create a notch in a bone tunnel during surgery, such as ligament reconstruction surgery, as will be further described below. The tap 20 includes a shaft 21 and a handle 22 coupled to a proximal portion 21a of the shaft 21. Additionally, the shaft 21 includes a distal portion 21b having threads 21b′ and configured for creating the notch in the bone tunnel, as will be further described below. The distal portion 21b also includes a laser mark 21b″ for cooperating with the laser mark 15 on the tip 13 in controlling the insertion depth of the tap 20 into the bone tunnel, as will be further described below. The handle 22 includes protrusions 22a that are configured for providing a user with a proper grip during creation of the notch.

As mentioned above, during ligament reconstruction surgery, the damaged ligament is removed and bone tunnels are created in the tibia and in the femur. A soft tissue graft, such as a patellar tendon or other soft tissue graft, is placed within the tunnels with one end of the graft being placed in the tibial tunnel and the other end of the graft being placed in the femoral tunnel. The ends may be fixated in the tunnels by a fixation device, such as a bone screw or other fixation device, by inserting the fixation device between walls of the tunnels and the soft tissue grafts. However, for appropriate insertion of the fixation device, the insertion sight must be properly prepared. Otherwise, cracking of the screws and/or twisting of the soft tissue graft may occur.

FIG. 5 shows the use of the tap guide 10 and the tap 20 for creating a notch in a wall of the bone tunnel 31 of a bone 30 during ligament reconstruction surgery, such as ligament reconstruction surgery. Such a notch creates enough of a pathway to substantially reduce the possibility of graft rotation and/or screw breakage, but allow enough contact between the screw and the graft for screw fixation. After creation of the bone tunnel 31 via a drill or other device for creating bone tunnels, the tip 13 of the tap guide 10 is inserted into the bone tunnel 31 until the laser mark 15 is aligned with the opening 31a to the tunnel 31. The tip 13 has a diameter that is substantially equal to the diameter of the bone tunnel 31. The diameter of the tip 13 is between about 4 mm and about 12 mm. After insertion of the tip 13, the tap 20 is then inserted into the through hole 11c of the shaft 11 and subsequently inserted into the bone tunnel 31 by rotating the tap 20 such that the threaded distal portion 21b of the shaft 21 creates a notch in the wall 32 of the bone tunnel 31. The tap 20 is rotated until the laser mark 21b″ of the tap 20 is aligned with the laser mark 15 of the tip 13. The threaded distal portion 21b extends about 2.5 cm along the length of the shaft 21. Consequently, the length of the notch is also about 2.5 cm. This length helps to substantially reduce screw breakage upon insertion of the screw into the bone tunnel 31 and allows rotation of the screw to occur the entire length, as will be more fully described below.

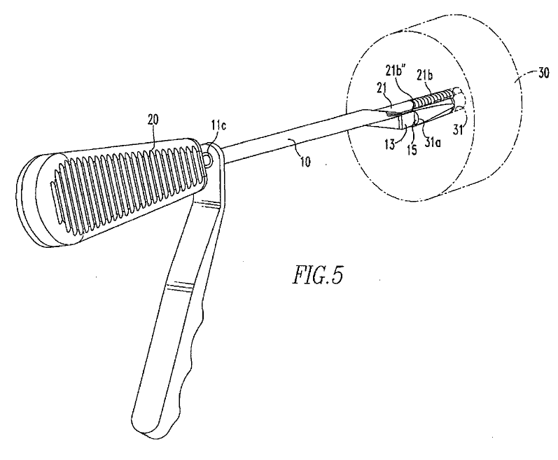

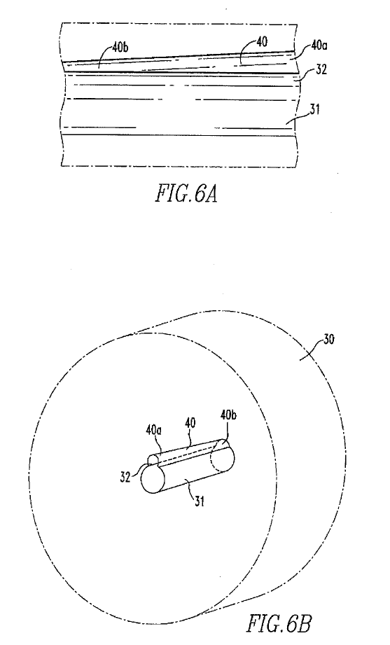

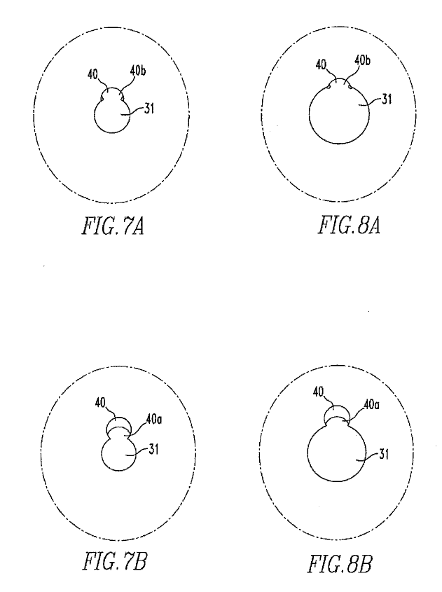

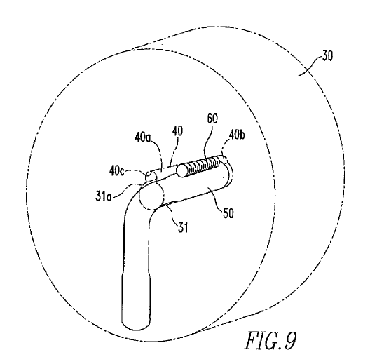

FIGS. 6A and 6B show the notch 40 that is created in the wall 32 of the bone tunnel 31. As stated above, the tip 13 includes a groove 14 having a tapered depth along a length of the groove 14. Consequently, during the creation of the notch 40, the distal portion 21b of the tap 20 cuts deeper in a first area 40a of the notch 40 than in a second area 40b of the notch 40. This difference in depth is more clearly shown in FIGS. 7A-7B and 8A-8B. The bone tunnels 31 in FIGS. 7A-B and 8A-B are 6 mm and 10 mm, respectively. A tap having a 4 mm diameter shaft was used to make the notch 40. FIGS. 7A and 8A show the notch 40 in the second area 40b of the notch 40 and FIGS. 7B and 8B show the notch 40 in the first area 40a of the wall 40.

After creation of the notch 40, the tip 13 of the tap guide 10 and the tap 20 are removed from the bone tunnel 31. As shown in FIG. 9, a tissue graft 50 is then inserted into the bone tunnel 31 and a fixation device 60, such as a bone screw, is inserted into bone tunnel 31, via the notch 40, and located between the tissue graft 50 and a wall 40c of the notch 40. The screw 60 is inserted via an insertion device. The screw 60 may be inserted into the bone tunnel 31 until an end of the screw 60 is flush with the opening 31a of the bone tunnel 31, however the depth of the screw 60 into the tunnel 31 will vary depending on surgical technique, as is shown in FIG. 9 where the screw 60 is located more towards the second area 40b of the notch 40. Having a notch 40 with a deeper first area 40a allows for easier insertion of the fixation device 60 into the tunnel 31 while still allowing enough contact between the device 60 and the soft tissue graft 50.

The shaft 11, handle 12, and tip 13 of the tap guide 10 are each made via a machining process, such as grinding, milling, reaming, ram, or wire EDM and include a biocompatible metal material, such as stainless steel or titanium alloy. However, other processes or materials may be used. The handle 12 and tip 13 are coupled to the shaft 11 via a soldering or welding process such as silver soldering, tig welding, laser welding, or any other type of soldering or welding process. In addition, a process other than soldering or welding may be used to couple the handle 12 and tip 13 to the shaft 11. The groove 14 in the tip 13 is made via a machining process, such as grinding, milling, reaming, ram, or wire EDM. However, other processes may be used.

The shaft 21 and handle 22 of the tap 20 are each made via a machining or sheet metal stamping process, such as grinding, milling, reaming, ram, or wire EDM and include a biocompatible metal material, such as stainless steel or titanium alloy. However, other processes or materials may be used. The threads 21b′ on the distal portion 21b of the shaft 21 are created via a machining process, such as grinding, milling, reaming, ram, or wire EDM. However, other processes may be used. The handle 22 of the tap 20 is coupled to the shaft 21 via a soldering or welding process such as silver soldering, tig welding, laser welding, or any other type of soldering or welding process. In addition, a process other than soldering or welding may be used to couple the handle 22 to the shaft 21.

As various modifications could be made to the exemplary embodiments, as described above with reference to the corresponding illustrations, without departing from the scope of the disclosure, it is intended that all matter contained in the foregoing description and shown in the accompanying drawings shall be interpreted as illustrative rather than limiting. Thus, the breadth and scope of the present disclosure should not be limited by any of the above-described exemplary embodiments, but should be defined only in accordance with the following claims appended hereto and their equivalents.

What is claimed is:

1. A tap guide comprising:
a shaft having a proximal portion and a distal portion;
a handle coupled to the proximal portion of the shaft; and
a tip coupled to the distal portion of the shaft.

2. The tap guide of claim 1 wherein the tip includes a groove.

3. The tap guide of claim 2 wherein the groove includes a tapered depth along a length of the groove.

4. The tap guide of claim 3 wherein the groove is between about 40% to about 65% deeper at a second end of the groove relative to a first end of the groove.

5. The tap guide of claim 1 wherein the tip is located at an angle α relative to a longitudinal axis of the shaft.

6. The tap guide of claim 5 wherein α is between about 0° to about 45°.

7. The tap guide of claim 1 wherein the tip includes a diameter of between about 4 mm to about 12 mm.

8. The tap guide of claim 1 wherein the shaft includes a through hole extending a length of the shaft.

9. The tap guide of claim 1 wherein the proximal portion includes a circular shape.

10. A method of preparing a bone tunnel for receipt of an anchor comprising:
providing a tap guide including a shaft having a proximal portion and a distal portion, a handle coupled to the proximal portion of the shaft, and a tip coupled to the distal portion of the shaft;
providing a tap;
inserting the tip into a bone tunnel;
inserting the tap into a through hole of the shaft and into the bone tunnel to create a notch in a wall of the bone tunnel; and
removing the tip from the bone tunnel.

11. The method of claim 10 further comprising:
inserting an end of a tissue graft into the bone tunnel;
inserting a fixation device into the bone tunnel via the notch, the fixation device located between the tissue graft and a wall of the notch.

12. The method of claim 10 wherein the tip includes a groove, the groove having a tapered depth along a length of the groove such that during creation of the notch the tap cuts deeper in a first area of the notch than in a second area of the notch.

13. The method of claim 10 wherein the diameter of the tip is substantially equal to the diameter of the bone tunnel.

14. A system for preparing a bone tunnel for receipt of a fixation device comprising:
a tap; and
a tap guide comprising a shaft having a proximal portion and a distal portion,
a handle coupled to the proximal portion of the shaft, and a tip coupled to the distal portion of the shaft.

15. The system of claim 14 wherein the tip includes a groove.

16. The system of claim 15 wherein the groove includes a tapered depth along a length of the groove.

17. The system of claim 16 wherein the groove is between about 40% to about 65% deeper at a second end of the groove relative to a first end of the groove.

18. The system of claim 14 wherein the tip is located at an angle α relative to a longitudinal axis of the shaft.

19. The system of claim 18 wherein α is between about 0° to about 45°.

20. The system of claim 14 wherein the tip includes a diameter of between about 4 mm to about 12 mm.

21. The system of claim 14 wherein the shaft includes a through hole extending a length of the shaft.

22. The system of claim 14 wherein the proximal portion includes a circular shape.

23. The tap guide of claim 1 wherein the handle includes a proximal portion and a distal portion, the distal portion located at an angle relative to the proximal portion, the proximal portion positioned substantially perpendicular to the proximal portion of the shaft.

24. The method of claim 11 wherein the notch is configured such that rotation of the tissue graft does not occur during insertion of the anchor into the bone tunnel.
